(19) United States
(12) Patent Application Publication
(10) Pub. No.: US 2023/0035668 A1
(43) Pub. Date: Feb. 2, 2023

(54) VISION TEST AND PROVISION OF PRESCRIPTION GLASSES

(71) Applicant: Snap Inc., Santa Monica, CA (US)

(72) Inventor: Brent Mills, Vashon, WA (US)

(21) Appl. No.: 17/815,887

(22) Filed: Jul. 28, 2022

Related U.S. Application Data
(60) Provisional application No. 63/203,734, filed on Jul. 29, 2021.

(51) Int. Cl. A61B 3/024; G06Q 30/06; G06T 19/00

(52) U.S. Cl. CPC A61B 3/024; G06Q 30/0643; G06T 19/006

(57) ABSTRACT

A method of providing prescription glasses to a user according to their vision prescription includes performing an eye test using a display device of a head-worn device to determine the user's vision prescription. A selection of available glasses frames are displayed on an associated portable device and user selection of a glasses frame is received using the portable device. An AR model of the selected glasses frame is displayed on an image of the user captured by a camera of the portable device. Final user selection of a glasses frame is received, and the user's vision prescription and an identifier of the finally-selected glasses frame are transmitted to at least one vendor of optical prescription services.

### RELATED APPLICATION DATA

This application claims the benefit of U.S. Provisional Patent Application No. 63/203,734, filed Jul. 29, 2021, the contents of which are included herewith by reference as if explicitly set forth.

### TECHNICAL FIELD

The present disclosure relates generally to wearable devices and to vision assessments performed using wearable devices.

### BACKGROUND

A head-worn device may be implemented with a transparent or semi-transparent display through which a user of the wearable device can view the surrounding environment. Such devices enable a user to see through the transparent or semi-transparent display to view the surrounding environment, and to also see objects (e.g., virtual objects such as 3D renderings, images, video, text, and so forth) that are generated for display to appear as a part of, and/or overlaid upon, the surrounding environment. This is typically referred to as “augmented reality.”

A head-worn device may additionally completely occlude a user's visual field and display a virtual environment through which a user may move or be moved. This is typically referred to as “virtual reality.” As used herein, the term “augmented reality” or “AR” refers to both augmented reality and virtual reality as traditionally understood, unless the context indicates otherwise.

### BRIEF DESCRIPTION OF THE SEVERAL VIEWS OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some nonlimiting examples are illustrated in the figures of the accompanying drawings in which:

FIG. 1 is a diagrammatic representation of a networked environment in which the present disclosure may be deployed, in accordance with some examples.

FIG. 2 is a diagrammatic representation of a messaging system, in accordance with some examples, that has both client-side and server-side functionality.

FIG. 3 is a perspective view of a wearable device, in accordance with some examples.

FIG. 4 is a block diagram illustrating a networked system including details of a wearable device, in accordance with one example.

FIG. 5 illustrates a wearable device (e.g., AR glasses 500) including displays 410 forming an integrated display, in accordance with some examples.

FIG. 6 illustrates variable focus lenses that may be included with the glasses of FIG. 3 or FIG. 5.

FIG. 7 is a schematic diagram illustrating the positioning of an augmented reality eye chart, according to one example.

FIG. 8A and FIG. 8B illustrate a flowchart 800 of a process for providing services relating to the provision of eye test and prescription glasses services, in accordance with one example.

FIG. 9 is a flowchart illustrating a process for determining a user's eyeglasses prescription, in accordance with one example.

FIG. 10 is a flowchart illustrating a process for conducting an AR eye test, in accordance with one example.

FIG. 11A is an example of a user interface that is displayed on a client device in response to receiving user selection of a glasses frame in the flowchart of FIG. 8A and FIG. 8B.

FIG. 11B is an example of a user interface that may be displayed in response to capture input in the user interface of FIG. 11A.

FIG. 12 is block diagram showing a software architecture within which the present disclosure may be implemented, in accordance with some examples.

FIG. 13 is a diagrammatic representation of a machine, in the form of a computer system within which a set of instructions may be executed for causing the machine to perform any one or more of the methodologies discussed, in accordance with some examples.

### DETAILED DESCRIPTION

According to some examples, a wearable device is implemented with a transparent or semi-transparent display enables a user to see through the transparent or semi-transparent display to view the surrounding environment. In addition, the wearable device may enable the user to see objects (e.g., virtual objects such as 3D renderings, images, video, text, and so forth) that are generated for display to appear as a part of, and/or overlaid upon, the surrounding environment. Such a wearable device may provide an augmented reality experience for the user.

The wearable device includes an integrated display for displaying objects (e.g., virtual objects) to the wearer of the device. The integrated display may for example include a waveguide that receives a light beam from a projector but any appropriate display for presenting augmented or virtual content to the wearer may be used.

The wearable device in use displays a virtual eye chart to the wearer, typically but not necessarily in conjunction with an associated portable computing device. The eye chart is rendered at the appropriate apparent distance and at the appropriate apparent size to present a standard eye chart, which can be used to assess the user's vision using familiar techniques, but without requiring a visit to the optometrist. Audio prompts may be provided to step the user through the eye chart, and the user's responses can be captured and recognized by the wearable device or the portable device using voice recognition techniques, hand gestures or through some other input device associated with the wearable device, such as a smartphone or other portable computing device. The responses by the user can be compared against expected responses to the prompts and the user's performance can be scored. An overall visual score can then be generated for each eye, which can be presented to the user or transmitted to a medical office. In one example, the visual score is a prescription for correction of the user's vision.

In some examples, the user may browse, select and purchase a prescription glasses frame using an online storefront, integrated with AR functionality to show the user what they look like wearing available frames. The selected frame and the user's glasses prescription may then be transmitted to one or more vendors to fulfill the order.

The wearable device may also comprise one or more cameras facing towards the wearer's eyes in use. These cameras may be used to image the wearer's eyes to determine if there may be any ocular conditions requiring further attention. Additionally, the wearable device may comprise one or more light emitters in the visual or near visual spectrum that may be used to illuminate an exterior or interior surface of the eye to assist with this determination. Furthermore, the light emitters may be configured to project a pattern onto the wearer's retinas, which can then be captured and analyzed by the cameras facing towards the wearer's eyes.

In some examples, provided is a method, executed by one or more processors, of providing prescription glasses to a user according to their vision prescription. The method includes performing an eye test using a display device of a head-worn device to determine the user's vision prescription, displaying a selection of glasses frames on a portable device, receiving user selection of a glasses frame using the portable device, displaying an AR model of the selected glasses frame on an image of the user captured by a camera of the portable device, receiving final selection of a glasses frame, and transmitting the user's vision prescription and an identifier of the finally selected glasses frame to at least one vendor of optical prescription services.

The method may further include providing a prompt via the portable device relating to whether or not the user has access to a head-worn device, and in response to receiving user input indicating that the user does not have access to a head-worn device, transmitting instructions to ship a head-worn device to the user. The method may further include obtaining a payment authorization prior to transmitting the instructions to ship a head-worn device to the user. In some examples, the method further includes, upon receiving notification of return of the head-worn device from the user, canceling or refunding the payment authorization.

A capture-media input may be received while the AR model is being displayed on an image of the user, media corresponding to the display of the AR model on the image of the user may be captured, and in response to receiving a forward-media input, the captured media may be transmitted in a message or chat session.

In some examples, the method may also include creating a joint communication session with a remote device associated with an eye-care practitioner, transmitting data relating to the eye test being performed using the display device of a head-worn device to the remote device, and receiving approval data for the user's vision prescription from the remote device. Summary data, generated during the performance of the eye test using the display device of a head-worn device eye test, may be transmitted to a remote device associated with an eye-care practitioner, and approval data for the user's vision prescription may be received from the remote device.

The method may further include displaying a user interface element listing characteristics of prescription glasses frames, receiving user selection of one or more specific characteristics, and limiting the selection of glasses frames on the portable device by the specific characteristics.

The method may also include further includes displaying identifiers of the limited selection of glasses frames in a carousel user interface, receiving touch input to place a particular glasses frame identifier in a central position in the carousel user interface, and displaying an AR model of the particular glasses frame on an image of the user captured by a camera of the portable device.

In some examples, provided is a non-transitory computer-readable storage medium, the computer-readable storage medium including instructions that when executed by a computer system, cause the computer system to provide prescription glasses to a user according to their vision prescription by performing operations according to any one of the method steps and limitations set forth above, including but not limited to performing an eye test using a display device of a head-worn device to determine the user's vision prescription, displaying a selection of glasses frames on a portable device, receiving user selection of a glasses frame using the portable device, displaying an AR model of the selected glasses frame on an image of the user captured by a camera of the portable device, receiving final selection of a glasses frame, and transmitting the user's vision prescription and an identifier of the finally selected glasses frame to at least one vendor of optical prescription services.

In some examples, provided is a computer system includes one or more processors and a memory storing instructions that, when executed by the one or more processors, configure the computer system to provide prescription glasses to a user according to their vision prescription by performing operations according to any one of the method steps and limitations set forth above, including but not limited to performing an eye test using a display device of a head-worn device to determine the user's vision prescription, displaying a selection of glasses frames on a portable device, receiving user selection of a glasses frame using the portable device, displaying an AR model of the selected glasses frame on an image of the user captured by a camera of the portable device, receiving final selection of a glasses frame, and transmitting the user's vision prescription and an identifier of the finally selected glasses frame to at least one vendor of optical prescription services.

Other technical features may be readily apparent to one skilled in the art from the following figures, descriptions, and claims.

Networked Computing Environment

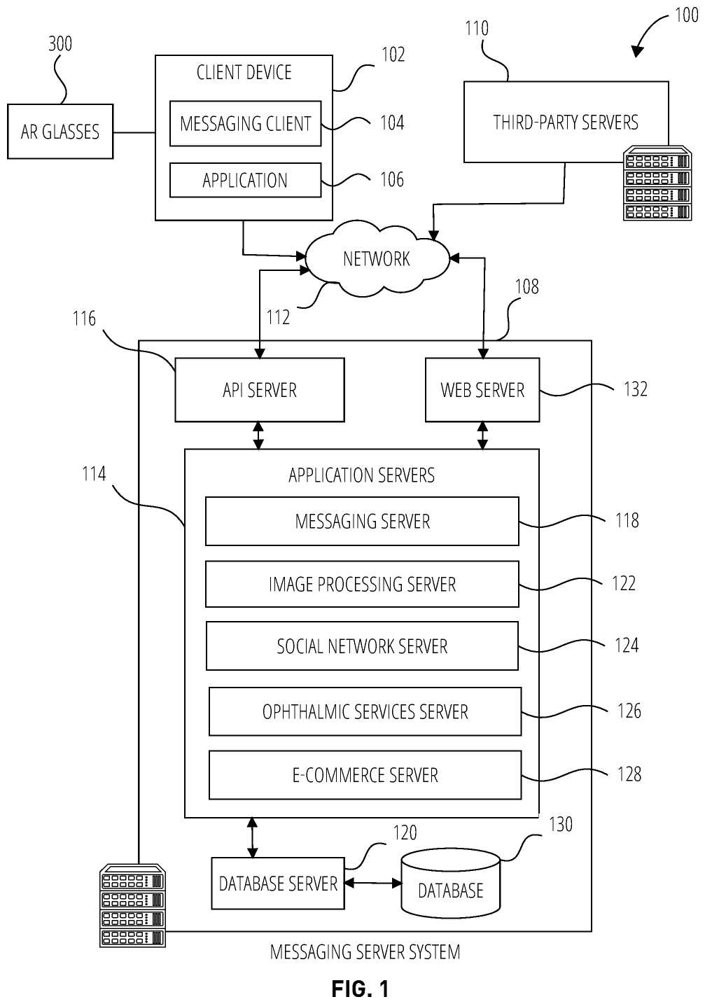

FIG. 1 is a block diagram showing an example messaging system 100 for exchanging data (e.g., messages and associated content) over a network. The messaging system 100 includes multiple instances of a user device 102, each of which hosts a number of applications, including a messaging client 104 and other applications 106. Each messaging client 104 is communicatively coupled to other instances of the messaging client 104 (e.g., hosted on respective other client devices 102), a messaging server system 108 and third-party servers 110 via a network 112 (e.g., the Internet). A messaging client 104 can also communicate with locally-hosted applications 106 using Applications Program Interfaces (APIs).

Also associated with one or more of the client devices 102 is a wearable device, comprising for example AR glasses 300/500/600 in some examples.

A messaging client 104 is able to communicate and exchange data with other messaging clients 104 and with the messaging server system 108 via the network 112. The data exchanged between messaging clients 104, and between a messaging client 104 and the messaging server system 108, includes functions (e.g., commands to invoke functions) as well as payload data (e.g., text, audio, video or other multimedia data).

The messaging server system 108 provides server-side functionality via the network 112 to a particular messaging client 104. While certain functions of the messaging system 100 are described herein as being performed by either a messaging client 104 or by the messaging server system 108, the location of certain functionality either within the messaging client 104 or the messaging server system 108 may be a design choice. For example, it may be technically preferable to initially deploy certain technology and functionality within the messaging server system 108 but to later migrate this technology and functionality to the messaging client 104 where a user device 102 has sufficient processing capacity.

The messaging server system 108 supports various services and operations that are provided to the messaging client 104. Such operations include transmitting data to, receiving data from, and processing data generated by the messaging client 104. This data may include message content, client device information, geolocation information, media augmentation and overlays, message content persistence conditions, social network information, and live event information, as examples. Data exchanges within the messaging system 100 are invoked and controlled through functions available via user interfaces (UIs) of the messaging client 104.

Turning now specifically to the messaging server system 108, an Application Program Interface (API) server 116 is coupled to, and provides a programmatic interface to, application servers 114. The application servers 114 are communicatively coupled to a database server 120, which facilitates access to a database 130 that stores data associated with messages processed by the application servers 114. Similarly, a web server 132 is coupled to the application servers 114, and provides web-based interfaces to the application servers 114. To this end, the web server 132 processes incoming network requests over the Hypertext Transfer Protocol (HTTP) and several other related protocols.

The Application Program Interface (API) server 116 receives and transmits message data (e.g., commands and message payloads) between the user device 102 and the application servers 114. Specifically, the Application Program Interface (API) server 116 provides a set of interfaces (e.g., routines and protocols) that can be called or queried by the messaging client 104 in order to invoke functionality of the application servers 114. The Application Program Interface (API) server 116 exposes various functions supported by the application servers 114, including account registration, login functionality, the sending of messages, via the application servers 114, from a particular messaging client 104 to another messaging client 104, the sending of media files (e.g., images or video) from a messaging client 104 to a messaging server 118, and for possible access by another messaging client 104, the settings of a collection of media data (e.g., story), the retrieval of a list of friends of a user of a user device 102, the retrieval of such collections, the retrieval of messages and content, the addition and deletion of entities (e.g., friends) to an entity graph (e.g., a social graph), the location of friends within a social graph, and opening an application event (e.g., relating to the messaging client 104).

The application servers 114 host a number of server applications and subsystems, including for example a messaging server 118, an image processing server 122, and a social network server 124. The messaging server 118 implements a number of message processing technologies and functions, particularly related to the aggregation and other processing of content (e.g., textual and multimedia content) included in messages received from multiple instances of the messaging client 104. As will be described in further detail, the text and media content from multiple sources may be aggregated into collections of content (e.g., called stories or galleries). These collections are then made available to the messaging client 104. Other processor and memory intensive processing of data may also be performed server-side by the messaging server 118, in view of the hardware requirements for such processing.

The application servers 114 also include an image processing server 122 that is dedicated to performing various image processing operations, typically with respect to images or video within the payload of a message sent from or received at the messaging server 118.

The social network server 124 supports various social networking functions and services and makes these functions and services available to the messaging server 118.

The ophthalmic services server 126 provides administrative services to the ophthalmic assessment and associate services described herein, including but not limited to maintaining associated records for users of the AR glasses 300 who have utilized the ophthalmic services described herein.

The e-commerce server 128 provides services relating to the selection, ordering and fulfillment of prescription glasses that have been ordered by a user based on the ophthalmic services described herein. These include providing glasses styles and options to a user who has or intends to take the visual acuity tests described herein, receiving selection of a particular style of glasses and any associated options such as frame color, lens tint, etc., receiving a final order and payment information, and placing an order for the prescription glasses with a manufacturer or other provider. The ecommerce server may also provide ordering and payment for the AR glasses 300 in response to a user who does not have the AR glasses 300 or has AR glasses that do not support the tests described herein. In one example, the AR glasses 300 are to be used for the tests described herein and then returned by the user once the tests and ordering have been complete. In such a case, the AR glasses 300 may be shipped to the user in operation 812 with a return-shipping label and an appropriate deposit taken, or other authorization provided, to ensure either return of the AR glasses 300 or purchase thereof if not returned by a certain deadline.

System Architecture

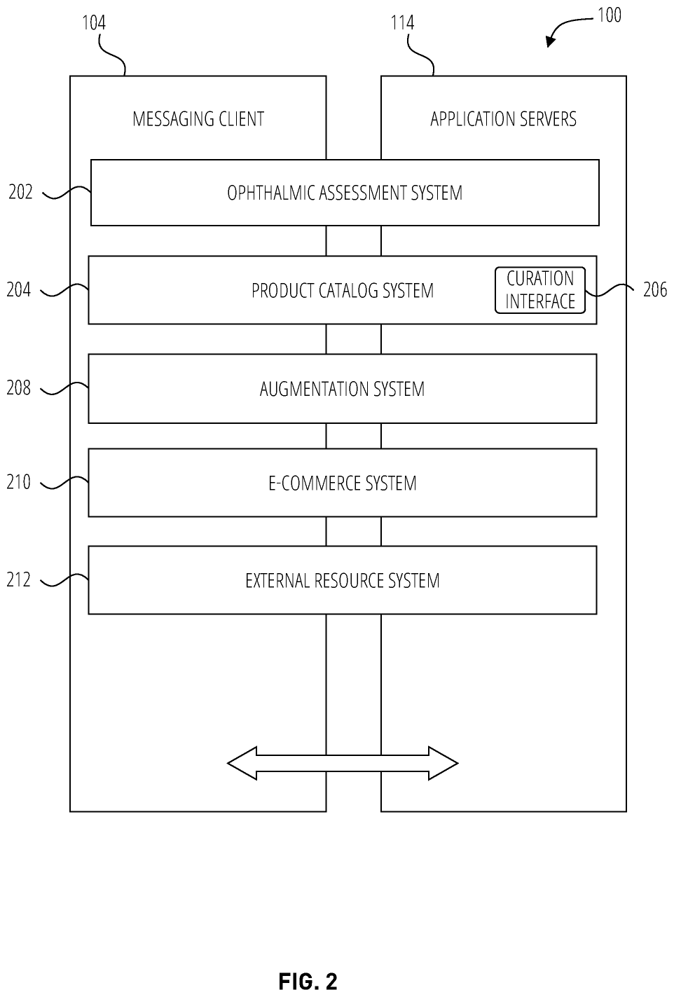

FIG. 2 is a block diagram illustrating further details regarding the messaging system 100, according to some examples. Specifically, the messaging system 100 is shown to comprise the messaging client 104 and the application servers 114. The messaging system 100 embodies a number of subsystems, which are supported on the client-side by the messaging client 104 and on the sever-side by the application servers 114. These subsystems include, for example, a product catalog system 204, an augmentation system 208, an ophthalmic assessment system 202, an e-commerce system 210, and an external resource system 212.

The product catalog system 204 is responsible for managing sets or collections of media (e.g., collections of text, image video, and audio data) relating to available brands, styles and options of prescription glasses available for purchase by the user. In particular, the product catalog system 204 may include collections of AR models of prescription glasses that can be applied by the augmentation system 208 to a live feed of a user captured by a camera in user device 102, or a still image or a video clip showing the face of a person such as the user of the device or a friend. A collection of content may be organized or searchable using metadata into various categories, e.g. based on gender identity, colors, styles, brands etc.

The product catalog system 204 furthermore includes a curation interface 206 that allows a collection manager to manage and curate a particular collection of content. For example, the curation interface 206 enables a prescription glasses brand company to access (from a third-party server 110) and curate a collection of content relating to their products, which is then available for viewing by a user of the user device 102.

The augmentation system 208 provides various functions that enable a user to augment (e.g., annotate or otherwise modify or edit) media content. For example, the augmentation system 208 provides functions related to the generation and publishing of media overlays content processed by the messaging system 100. A media overlay may include audio and visual content and visual effects. Examples of audio and visual content include pictures, texts, logos, animations, and sound effects. The audio and visual content or the visual effects can be applied to a media content item (e.g., a live video feed, photo or video clip) at the user device 102.

In the example described herein, the augmentation system 208 applies AR effect media overlays corresponding to a selected prescription glasses frame, to a face of a user or their friend, to provide a preview of their appearance when wearing the glasses. The augmentation system 208 also typically provides a user-based publication platform that enables users to publish images or video clips of the applied media overlay in a chat session or on a story feed associated with the user, to allow others to respond or comment on the user's appearance with the selected glasses.

The ophthalmic assessment system 202 provides the methods relating to ophthalmic assessments as described in more detail below, and, in conjunction with the ophthalmic services server 126, provides administrative services to the ophthalmic assessments, including but not limited to maintaining associated assessment records, prescription glasses preferences and selections, and so forth, for users of the AR glasses 300 who have utilized the ophthalmic services described herein.

The e-commerce system 210, in conjunction with the e-commerce server 128, provides services relating to the selection, ordering and fulfillment of prescription glasses that have been ordered by a user based on the ophthalmic services described herein.

In some examples, the methods described herein are integrated with a messaging client 104 or other standalone application 106. In other cases, the method described herein may be provided by applets or other third party resources that are accessed within the context of the messaging client 104 or other standalone application. For example, the product catalog system 204, ophthalmic assessment system 202 and e-commerce system 210 may all or individually be provided as an applet that is accessed within the context of the messaging client 104 or other standalone application 106.

The external resource system 212 provides an interface for the messaging client 104 to communicate with remote servers (e.g. third-party servers 110) to launch or access external resources such as applications or applets. Each third-party server 110 hosts, for example, a markup language (e.g., HTML5) based application or small-scale version of an application. The messaging client 104 may launch a web-based resource (e.g., application) by accessing the HTML5 file from the third-party servers 110 associated with the web-based resource. In certain examples, applications hosted by third-party servers 110 are programmed in JavaScript leveraging a Software Development Kit (SDK) provided by the messaging server 118. The SDK includes Application Programming Interfaces (APIs) with functions that can be called or invoked by the web-based application. In certain examples, the messaging server 118 includes a JavaScript library that provides a given external resource access to certain user data of the messaging client 104. HTML5 is used as an example technology, but applications and applets may be based on other technologies.

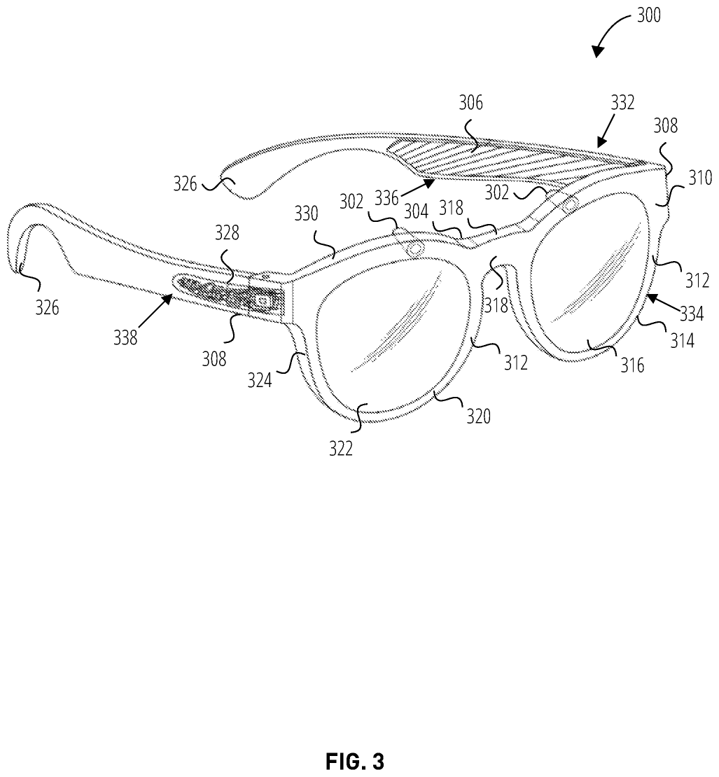

FIG. 3 is perspective view of a wearable device (e.g., AR glasses 300), in accordance with some examples. The AR glasses 300 can include a frame 332 made from any suitable material such as plastic or metal, including any suitable shape memory alloy. In one or more examples, the frame 332 includes a front piece 334 including a first or left optical element holder 314 (e.g., a display or lens holder) and a second or right optical element holder 320 connected by a bridge 318. The front piece 334 additionally includes a left end portion 310 and a right end portion 324. A first or left optical element 316 and a second or right optical element 322 can be provided within respective left optical element holder 314 and right optical element holder 320. Each of the right optical element 322 and the left optical element 316 can be a lens, a display, a display assembly, or a combination of the foregoing. Any suitable display assembly can be provided in the AR glasses 300.

The frame 332 additionally includes a left arm or temple piece 336 and a right arm or temple piece 338 coupled to the respective left end portion 310 and the right end portion 324 of the front piece 334 by any suitable means such as a hinge (not shown), so as to be coupled to the front piece 334, or rigidly or fixably secured to the front piece 334 so as to be integral with the front piece 334. In one or more implementations, each of the temple piece 336 and the temple piece 338 includes a first portion 308 that is coupled to the respective left end portion 310 or right end portion 324 of the front piece 334 and any suitable second portion 326 for coupling to the ear of the user. In one example, the front piece 334 can be formed from a single piece of material, so as to have a unitary or integral construction. In one example, such as illustrated in FIG. 3, the entire frame 332 can be formed from a single piece of material so as to have a unitary or integral construction.

The AR glasses 300 can include a computing device, such as a computer 328, which can be of any suitable type so as to be carried by the frame 332 and, in one or more examples, of a suitable size and shape, so as to be at least partially disposed in one of the temple piece 336 and the temple piece 338. In one or more examples, as illustrated in FIG. 3, the computer 328 is sized and shaped similar to the size and shape of one of the temple piece 338 (e.g., or the temple piece 336), and is thus disposed almost entirely if not entirely within the structure and confines of such temple piece 338. In one or more examples, the computer 328 is disposed in both of the temple piece 336 and the temple piece 338. The computer 328 can include one or more processors with memory, wireless communication circuitry, and a power source. As discussed below, the computer 328 comprises low-power circuitry, high-speed circuitry, and a display processor. Various other examples may include these elements in different configurations or integrated together in different ways. Additional details of aspects of computer 328 may be implemented as illustrated by the data processor 402 discussed below.

The computer 328 additionally includes a battery 306 or other suitable portable power supply. In one example, the battery 306 is disposed in one of the temple piece 336 or the temple piece 338. In the AR glasses 300 shown in FIG. 3, the battery 306 is shown as being disposed in left temple piece 336 and electrically coupled using the connection 330 to the remainder of the computer 328 disposed in the right temple piece 338. The AR glasses 300 can include a connector or port (not shown) suitable for charging the battery 306 accessible from the outside of frame 332, a wireless receiver, transmitter or transceiver (not shown), or a combination of such devices.

In one or more implementations, the AR glasses 300 include cameras 302. Although two cameras are depicted, other examples contemplate the use of a single or additional (i.e., more than two) cameras. In one or more examples, the AR glasses 300 include any number of input sensors or peripheral devices in addition to the cameras 302. The front piece 334 is provided with an outward facing, forward-facing or front or outer surface 312 that faces forward or away from the user when the AR glasses 300 are mounted on the face of the user, and an opposite inward-facing, rearward-facing or rear or inner surface 304 that faces the face of the user when the AR glasses 300 are mounted on the face of the user. Such sensors can include inwardly-facing video sensors or digital imaging modules such as cameras be mounted on or provided within the inner surface 304 of the front piece 334 or elsewhere on the frame 332 so as to be facing the user, and outwardly-facing video sensors or digital imaging modules such as the cameras 302 that can be mounted on or provided with the outer surface 312 of the front piece 334 or elsewhere on the frame 332 so as to be facing away from the user. Such sensors, peripheral devices or peripherals can additionally include biometric sensors, location sensors, or any other such sensors.

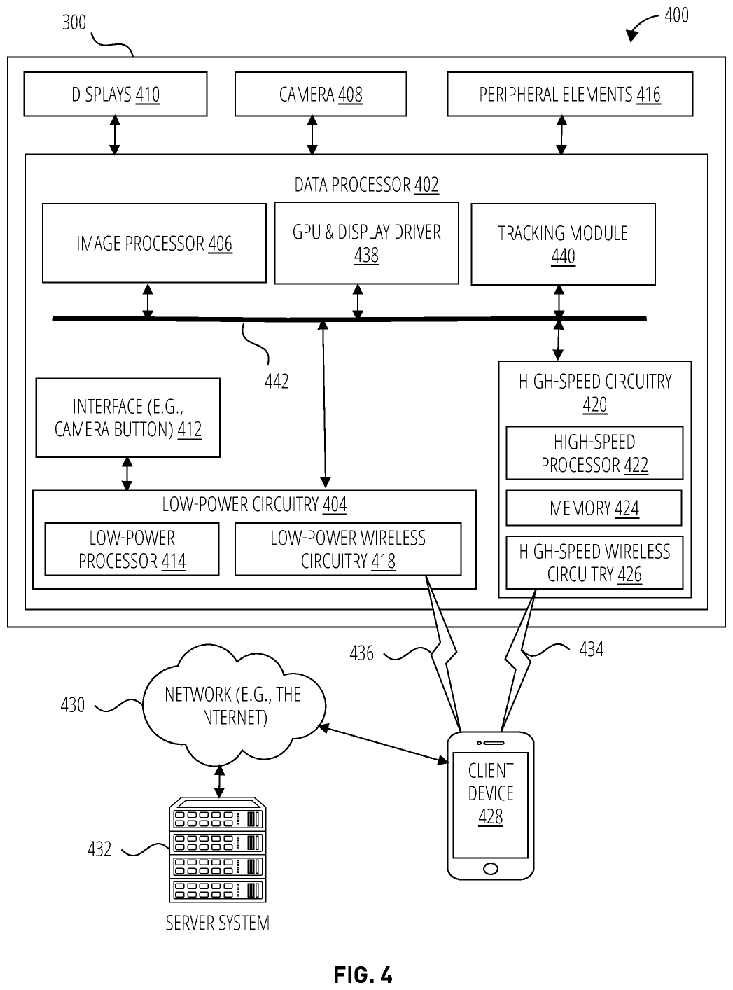

FIG. 4 is a block diagram illustrating a networked system 400 including details of the AR glasses 300, in accordance with some examples.

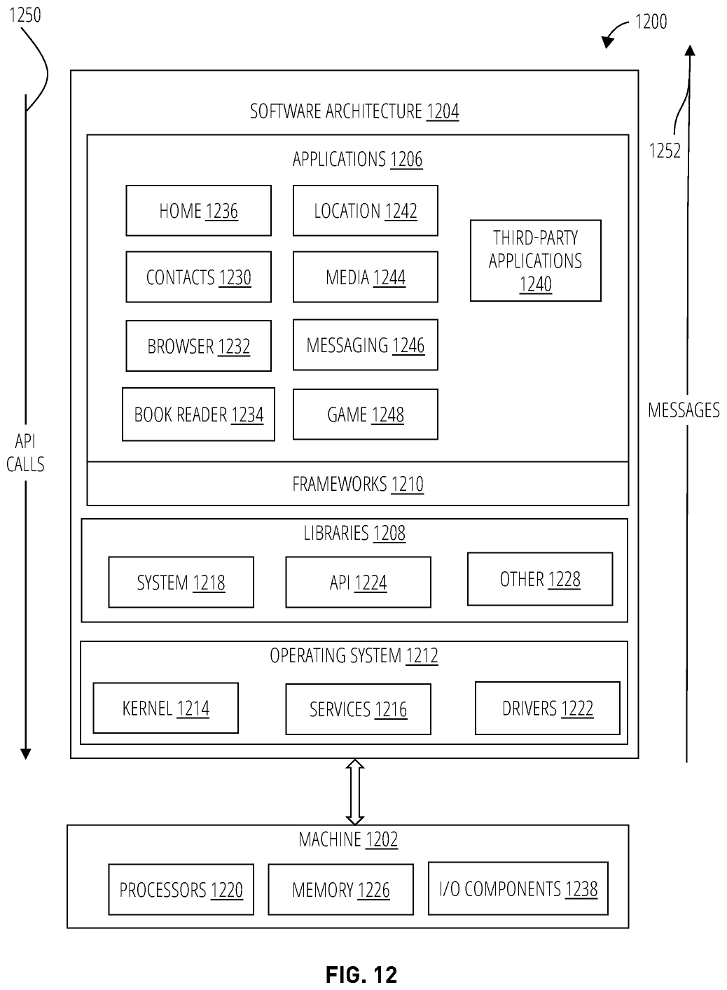

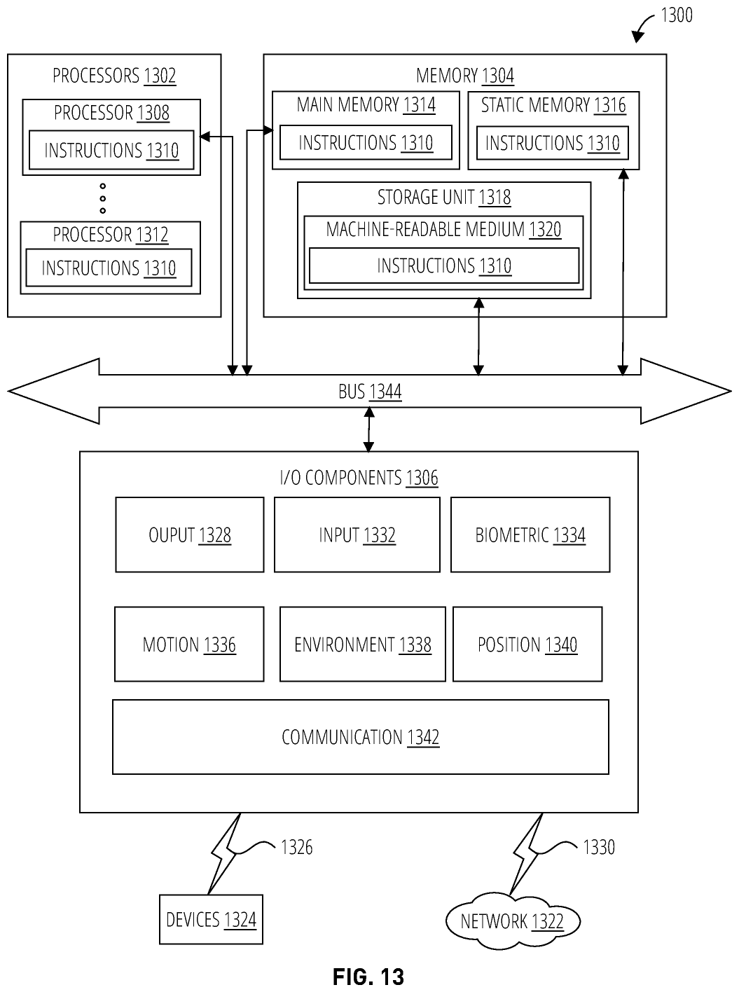

The networked system 400 includes the AR glasses 300, a client device 428, and a server system 432. The client device 428 may be a smartphone, tablet, phablet, laptop computer, access point, or any other such device capable of connecting with the AR glasses 300 using both a low-power wireless connection 436 and a high-speed wireless connection 434. The client device 428 is connected to the server system 432 via the network 430. The network 430 may include any combination of wired and wireless connections. The server system 432 may be one or more computing devices as part of a service or network computing system. The client device 428 and any elements of the server system 432 and network 430 may be implemented using details of the software architecture 1204 or the machine 1300 described in FIG. 12 and FIG. 13.

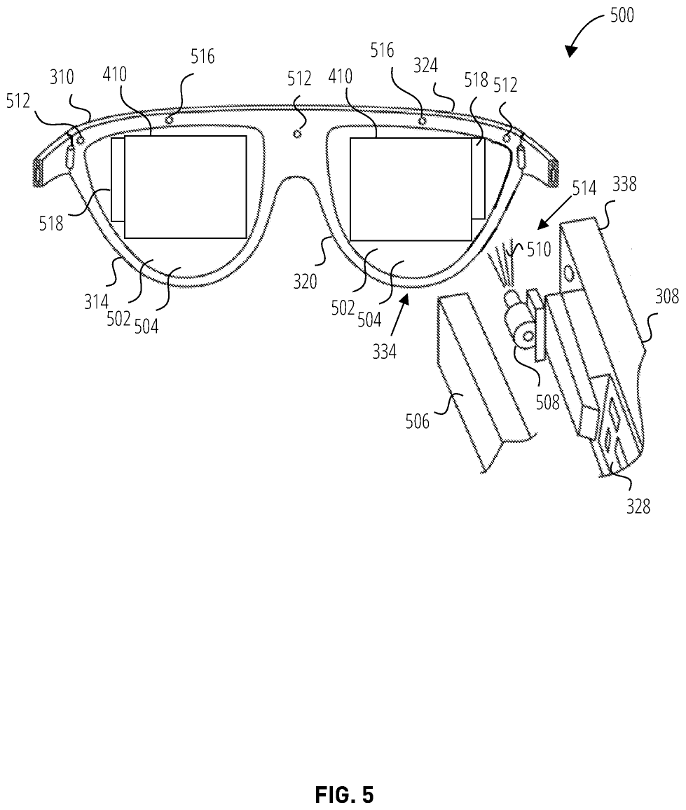

The AR glasses 300 include a data processor 402, displays 410, one or more cameras 408, additional peripheral elements 416. The peripheral elements 416 may include microphones, audio speakers, biometric sensors, additional sensors, or additional display elements integrated with the data processor 402. Examples of the peripheral elements 416 are discussed further with respect to FIG. 12 and FIG. 13. For example, the peripheral elements 416 may include any I/O components 1306 including output components 1328, motion components 1336, or any other such elements described herein. Examples of the displays 410 is discussed in FIG. 5. In the particular examples described herein, the displays 410 include a display for each one of a user's left and right eyes.

The data processor 402 includes an image processor 406 (e.g., a video processor), a GPU & display driver 438, a tracking module 440, an interface 412, low-power circuitry 404, and high-speed circuitry 420. The components of the data processor 402 are interconnected by a bus 442.

The interface 412 refers to any source of a user command that is provided to the data processor 402. In one or more examples, the interface 412 is a physical button on a camera that, when depressed, sends a user input signal from the interface 412 to a low-power processor 414. A depression of such a camera button followed by an immediate release may be processed by the low-power processor 414 as a request to capture a single image. A depression of such a camera button for a first period of time may be processed by the low-power processor 414 as a request to capture video data while the button is depressed, and to cease video capture when the button is released, with the video captured while the button was depressed stored as a single video file. In other examples, the interface 412 may be any mechanical switch or physical interface capable of accepting user inputs associated with a request for data from the camera 408. In other examples, the interface 412 may have a software component, or may be associated with a command received wirelessly from another source, such as from the client device 428.

The image processor 406 includes circuitry to receive signals from the camera 408 and process those signals from the camera 408 into a format suitable for storage in the memory 424 or for transmission to the client device 428. In one or more examples, the image processor 406 (e.g., video processor) comprises a microprocessor integrated circuit (IC) customized for processing sensor data from the camera 408, along with volatile memory used by the microprocessor in operation.

The low-power circuitry 404 includes the low-power processor 414 and the low-power wireless circuitry 418. These elements of the low-power circuitry 404 may be implemented as separate elements or may be implemented on a single IC as part of a system on a single chip. The low-power processor 414 includes logic for managing the other elements of the AR glasses 300. As described above, for example, the low-power processor 414 may accept user input signals from the interface 412. The low-power processor 414 may also be configured to receive input signals or instruction communications from the client device 428 via the low-power wireless connection 436. The low-power wireless circuitry 418 includes circuit elements for implementing a low-power wireless communication system. Bluetooth™ Smart, also known as Bluetooth™ low energy, is one standard implementation of a low power wireless communication system that may be used to implement the low-power wireless circuitry 418. In other examples, other low power communication systems may be used.

The high-speed circuitry 420 includes a high-speed processor 422, a memory 424, and a high-speed wireless circuitry 426. The high-speed processor 422 may be any processor capable of managing high-speed communications and operation of any general computing system needed for the data processor 402. The high-speed processor 422 includes processing resources needed for managing high-speed data transfers on the high-speed wireless connection 434 using the high-speed wireless circuitry 426. In certain examples, the high-speed processor 422 executes an operating system such as a LINUX operating system or other such operating system such as the operating system 1212 of FIG. 12. In addition to any other responsibilities, the high-speed processor 422 executing a software architecture for the data processor 402 is used to manage data transfers with the high-speed wireless circuitry 426. In certain examples, the high-speed wireless circuitry 426 is configured to implement Institute of Electrical and Electronic Engineers (IEEE) 802.11 communication standards, also referred to herein as Wi-Fi. In other examples, other high-speed communications standards may be implemented by the high-speed wireless circuitry 426.

The memory 424 includes any storage device capable of storing camera data generated by the camera 408 and the image processor 406. While the memory 424 is shown as integrated with the high-speed circuitry 420, in other examples, the memory 424 may be an independent standalone element of the data processor 402. In certain such examples, electrical routing lines may provide a connection through a chip that includes the high-speed processor 422 from image processor 406 or the low-power processor 414 to the memory 424. In other examples, the high-speed processor 422 may manage addressing of the memory 424 such that the low-power processor 414 will boot the high-speed processor 422 any time that a read or write operation involving the memory 424 is needed.

The tracking module 440 estimates a pose of the AR glasses 300. For example, the tracking module 440 uses image data and corresponding inertial data from the camera 408 and the position components 1340, as well as GPS data, to track a location and pose of the AR glasses 300 relative to a frame of reference (e.g., real-world environment). In one example, the tracking module 440 uses the sensor and image data to determine the three-dimensional pose of the AR glasses 300. The three-dimensional pose is a determined orientation and position of the AR glasses 300 in relation to the user's real-world environment. For example, tracking module 440 may use images of the user's real-world environment, as well as other sensor data, to identify a relative position and orientation of the AR glasses 300 relative to physical objects in the real-world environment surrounding the AR glasses 300 using techniques such as Structure from Motion or by using an existing 3D point cloud. The tracking module 440 continually gathers and uses updated sensor data describing movements of the AR glasses 300 to determine updated three-dimensional poses of the AR glasses 300 that indicate changes in the relative position and orientation relative to physical objects in the real-world environment. The tracking module 440 permits visual placement of virtual objects relative to physical objects by the AR glasses 300 within the field of view of the user via the displays 410.

The GPU & display driver 438 uses the three-dimensional pose of the AR glasses 300 to generate frames of virtual content to be presented on the displays 410. For example, the GPU & display driver 438 uses the three-dimensional pose to render a frame of virtual content such that the virtual content is presented at an appropriate orientation, position and depth in the displays 410, thereby to properly integrate the virtual content with the user's view of physical objects. The GPU appropriately scales and locates the virtual content so that the perceived size of the virtual content is consistent with the intended distance of the virtual content from the user. The GPU & display driver 438 generates updated frames of virtual content based on updated three-dimensional poses of the AR glasses 300, which reflect changes in the position and orientation of the user in relation to physical objects in the user's real-world environment.

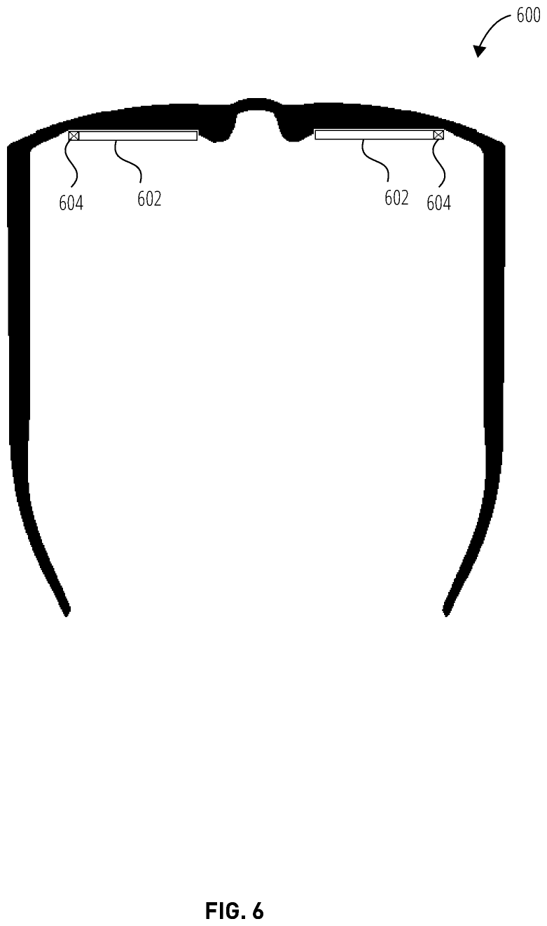

Virtual objects displayed in augmented or virtual reality environments are typically displayed at a fixed focal distance, typically infinity. That is, the light rays entering the eye from any particular virtual object are parallel (for an infinite focal distance), irrespective of the perceived or virtual distance of the object based on cues such as the size of the object, its placement with respect to other objects, and stereoscopic differences between the images presented to the left and right eyes. However, conventional eye tests are typically located at a distance of 20 ft (6m). In some cases, the AR glasses 300 may be provided with variable focus lenses see FIG. 6 below) to change the focal distance to the correct focal distance from whatever standard or default focal distance is provided in the head-worn device. In other cases, the existing focal distance provided by the AR glasses 300 or other head-worn device might provide sufficiently accurate eye test results even if the focal distance provided by the head-worn device does not correspond to the focal distance of a particular eye test.

One or more functions or operations described herein may also be performed in an application resident on the AR glasses 300 or on the client device 428, or on a remote server. For example, one or more functions or operations described herein may be performed by one of the applications 1206 such as messaging application 1246 or a custom eye test application.

FIG. 5 illustrates a wearable device (e.g., AR glasses 500) including displays 410 forming an integrated display, in accordance with some examples. The AR glasses 500 can be of any suitable type, including the AR glasses 300, and like reference numerals have been used to describe like components of AR glasses 500 and AR glasses 300. The AR glasses 500 include optical lenses 502 secured within each of the left optical element holder 314 and the right optical element holder 320. Each of the optical lenses 502 has a respective front surface (not shown) and an opposite rear surface 504.

The AR glasses 500 include an optical assembly 514 for displaying images to a user. In the example of FIG. 5, one optical assembly 514 is shown, but optical assemblies are normally provided for both eyes of a user (e.g., for both of the temple piece 336 and the temple piece 338). The optical assembly 514 includes an optical source such as a projector 508 that is disposed in one or both of the arms or temples of the glasses (e.g., the right temple piece 336 and left temple piece 338 of the AR glasses 500). The projector 508 may be covered in use by a cover 506.

The optical assembly 514 further includes displays 410. The displays 410 each include diffractive structures or a waveguide (e.g., gratings and/or optical elements). The displays 410 may also include an optical in-coupling element 518, such as a prism or mirror, located along the edge of the displays 410. The in-coupling element 518 is used to direct light 510 emitted by the projector 508 into the displays 410, where it encounters the diffractive structures of the displays 410. The diffractive structures direct the light 510 towards the eye of the user, thereby to provide an image on the rear surface 504 of the optical lens 502 that overlays the view of the real world seen by the user.

The AR glasses 500 further include inwardly-facing cameras 512, which are directed at the user's eye. Also provided are one or more light emitters that are similarly directed towards and can illuminate a user's pupil and retina. In some examples, the light emitters 516 are LED light emitters that emit an optical spectrum that is appropriate for illuminating the eye for capture of an image of the retina or pupil by the cameras 512 that can be used to identify problems or potential problems with the eye, for example glaucoma or the presence of cataracts in the lens of the user's eye. In other examples, the light emitters 516 may include a grid that projects a corresponding grid onto the user's retina. An image of the grid as projected onto the retina can then be captured by one or more of the cameras 512, which can be used to evaluate the user's visual acuity by comparison with a reference or expected appearance of the grid. In yet further examples, the light emitters 516 may be tiny projectors, which can either project a spectrum of light or an image, for example, a grid, onto the user's retina. As will be appreciated each of the cameras 512 and light emitters 516 are separately addressable to provide functionality that will vary depending on which eye is under test and the nature of the test.

In another implementation, the projector 508 can be used to project a light field into displays 410, which can provide sufficient illumination to permit the capture of images of the pupil or lens or retina of the pupil's eye by the cameras 512, which can be used to identify problems or potential problems with the eye, for example glaucoma or the presence of cataracts in the lens of the user's eye. The light field projected by the projectors 508 (one for each eye) and displayed by displays 410 may also for example include one or more features, such as a grid, the projection of which onto the user's retina can be used to evaluate the user's visual acuity by comparison with a reference or expected appearance of the grid.

While the cameras 512 and light emitters 516 are shown along an upper edge of the frame of the glasses, it will be appreciated that cameras 512 and light emitters 516 may be positioned elsewhere on the frame 332, for example around the left optical element holder 314 and right optical element holder 320. One or more of the cameras 512 on one or the other of the left optical element holder 314 or right optical element holder 320 may capture simultaneous or near simultaneous images, from which a composite image of one or more features of a user's eye may be constructed.

FIG. 6 illustrates variable focus lenses that may be included with AR glasses 300 or AR glasses 500. AR glasses 600 illustrated in FIG. 6 include left and right variable focus lenses 602. The variable focus lenses 602 are located between the displays 410 and optical lenses 502 and the user's eye, so that the variable focus lenses 602 operate both on the incident light received from the real world as well as on the light provided by the displays 410 (see FIG. 5). The variable focus lenses 602 also include actuators 604 for effecting changes in the focal lengths of the variable focus lenses 602.

The variable focus lenses 602 may be any type of variable focus lens, but in some examples may for example be a liquid lens, in which fluid pressure is varied to change the curvature of an elastic membrane, an Alvarez lens, in which the relative positions of two or more lenses can be varied to change the optical power of the lens, or a liquid crystal variable focus lens, in which the orientation of liquid crystal molecules can be varied to change the optical power of the lens.

Each variable focus lenses 602 may also include a plurality of variable focus elements arranged in series on an optical axis to provide the required variation in optical power. For example, a first variable focus element may be provided with a variable spherical base strength for correcting a user's distance vision and a second variable focus element may be provided with a variable cylinder base strength and variable axis (such as by permitting rotation of the second variable focus element around the optical axis in the case of Alvarez or fluid lenses), for correcting astigmatism. Alternatively, the variable base strength, variable cylinder strength and variable axis may be provided in a single LCD variable focus lens.

The actuators 604 provide the required input to change the optical power of the variable focus lenses 602. The nature of the actuators 604 will depend on the nature of the variable focus lenses 602 and may for example be a display driver in the case of an LCD variable focus lens, control electronics and an impeller or variable volume reservoir in the case of a fluid lens, or control electronics and mechanical actuators in the case of an Alvarez lens. Additionally, the actuators 604 may provide rotational motion between lens elements to provide variable base and cylindrical strengths, as well as rotation of a variable cylindrical strength element for cylindrical strength axis adjustment. The left and right variable focus lenses 602 are independently controllable to provide independent optical adjustment to the user's left and right eyes.

In use, the actuators 604 are controlled by signals received from the data processor 402 or the client device 428, or a combination thereof, to vary the optical power of each of the left and right variable focus lenses 602 as will be discussed in more detail below.

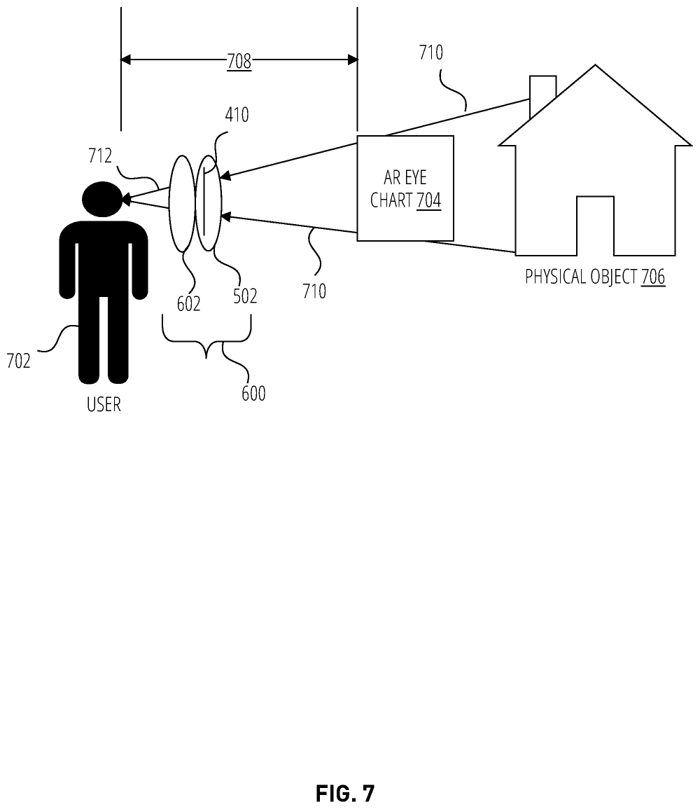

FIG. 7 is a schematic diagram illustrating the positioning of an augmented reality eye chart, according to one example. A user 702 is wearing AR glasses 600. Due to the transparent nature of the optical lenses 502, the displays 410 and the variable focus lenses 602, the user will perceive objects in the real world such as physical object 706 due to light rays 710 passing through the optical elements of the AR glasses 600 and into the user's eye. As will be described in more detail below, the user 702 has selected a mode in which a virtual AR eye chart 704 is displayed to the user 702 via the displays 410, for example comprising the left or right projectors 508 and the associated displays 410 in the optical lenses 502.

The output of the displays 410 combines with any of the rays 710 passing through the optical lens 502. The combined light rays then pass through the left and right variable focus lenses 602 to form rays 712, which provide a display to the user 702 in which the AR eye chart 704 overlays the physical object 706 at a perceived distance 708 and size that is appropriate for an eye test, and at a focal distance (provided by the variable focus lenses 602) that is appropriate for the eye test.

As is conventional, the AR eye chart 704 includes rows of letters of diminishing size, which the user can progressively attempt to read to assess visual acuity. In one example, the AR eye chart 704 is displayed on the display corresponding to the eye under test, while the other display shows nothing (i.e. the view through that optical lens 502 is unobstructed) or the display projects a uniform color such as black so that the eye that is not under test is unable to see beyond the corresponding optical lens 502.

In some examples, the location of the AR eye chart 704 is maintained by the tracking module 440 and GPU & display driver 438 in a persistent location relative to objects in the real world, for example physical object 706, so that when the user moves their head the appearance of the AR eye chart 704 remains fixed relative to real world objects. This is however may not be required as long as the perceived distance 708 from the AR eye chart 704 to the user is maintained. In such a case, the AR eye chart 704 will be fixed relative to the AR glasses 600 instead of with respect to a physical object 706. In another example, the AR eye chart 704 may be shown to appear, and be maintained, on a surface of an object in the real world, such as a wall, provided that such projection does not create a distracting inconsistency, for example if the wall is insufficiently parallel to the plane of the AR eye chart 704 or is not at the correct distance.

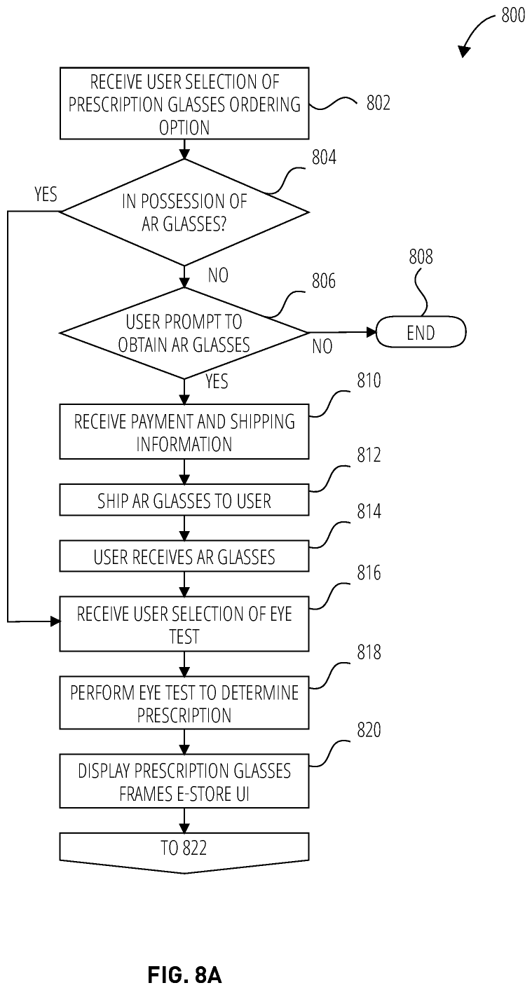

FIG. 8A and FIG. 8B illustrate a flowchart 800 of a process for providing services relating to the provision of eye test and prescription glasses services, in accordance with one example. For explanatory purposes, the operations of the flowchart 800 are described herein as occurring in serial, or linearly. However, multiple operations of the flowchart 800 may occur in parallel. In addition, the operations of the flowchart 800 need not be performed in the order shown and/or one or more blocks of the flowchart 800 need not be performed and/or can be replaced by other operations.

The operations illustrated in FIG. 8A will typically execute on a combination of the devices and systems described herein, as will be apparent from the description below of the flowchart 800. Variations are of course possible, depending on the implementation. For example, the ophthalmic services server 126, the e-commerce server 128 and the product catalog system 204 with collections of AR models of glasses frames could be hosted by one or more third parties (that is, not the provider of the messaging system 100) distributed across one or more third-party servers 110.

Various input points may be provided for the flowchart 800. For example, the user may be accessing and virtually trying on a selection of prescription glasses frames just for fun before deciding to order prescription glasses, they may previously have done an eye assessment as described herein first before deciding to order prescription glasses, or they may expressly have started with user selection of an option in messaging application 1246 to order prescription glasses from the start. Flowchart 800 describes the latter situation, but the order of the events will vary depending on the particular entry point, for example selection of a frame before conducting the eye assessment, and so forth. Also, the order of events will vary. For example, if AR glasses 600 are shipped to the user to permit the eye test to be performed. The user may browse and select and order prescription glasses frames in advance of receiving the AR glasses 600.

The flowchart 800 begins at operation 802 with the messaging application 1246 (or other application, or applet executing within the context of messaging application 1246) receiving user selection of an option to order prescription glasses. At operation 804 a check is done to determine if the user is in possession of appropriate AR glasses 600 with which to perform a vision assessment. This may be done automatically by the messaging application 1246 or user device 102 checking to see if AR glasses 600 are paired or associated with the user device 102. If the user is in possession of AR glasses 600, the method continues at operation 816.

If the user is not in possession of appropriate AR glasses 600 in operation 804, the method proceeds at operation 806 in which the messaging application 1246 provides a prompt for the user to obtain AR glasses 600. For example, the messaging application 1246 may provide a user prompt stating that AR glasses 600 are required to continue and asking the user if they would like to buy or rent AR glasses 600 for this purpose.

If the user refuses at operation 806, the method terminates at operation 808 or alternatively the user may be given the option to browse and try on frames in the curation interface 206 instead (see further FIG. 8B operation 822 to operation 834), which may encourage them to return at a later time to obtain the AR glasses 600 to do the required eye test for the purpose of obtaining prescription glasses.

If the user accepts the prompt to obtain AR glasses 600 at operation 810, the e-commerce system 210 running for example on e-commerce server 128 receives and processes payment and shipping information from the user, and passes an order for the AR glasses 600 to a fulfillment service for shipping and handling. The particular terms of the order will depend on whether the user is buying the AR glasses 600 or obtaining them temporarily for use in the method of FIG. 8A and FIG. 8B. In the latter case, a deposit or authorization or other hold may be placed against the user's payment information pending return of the AR glasses 600. In the event the AR glasses 600 are not returned within an appropriate time, the cost of the AR glasses 600 may be deducted, after appropriate notifications to the user, from the user's payment information. There may be a fee associated with rental or shipping of the glasses, and a return shipping label may be included with the AR glasses 600 to facilitate ease of return.

After receiving shipping and payment information in operation 810 and before receiving the AR glasses 600, the user may be passed to operation 822 to browse and select a prescription glasses frame. After the user receives the AR glasses 600 in operation 814, the method continues at operation 818.

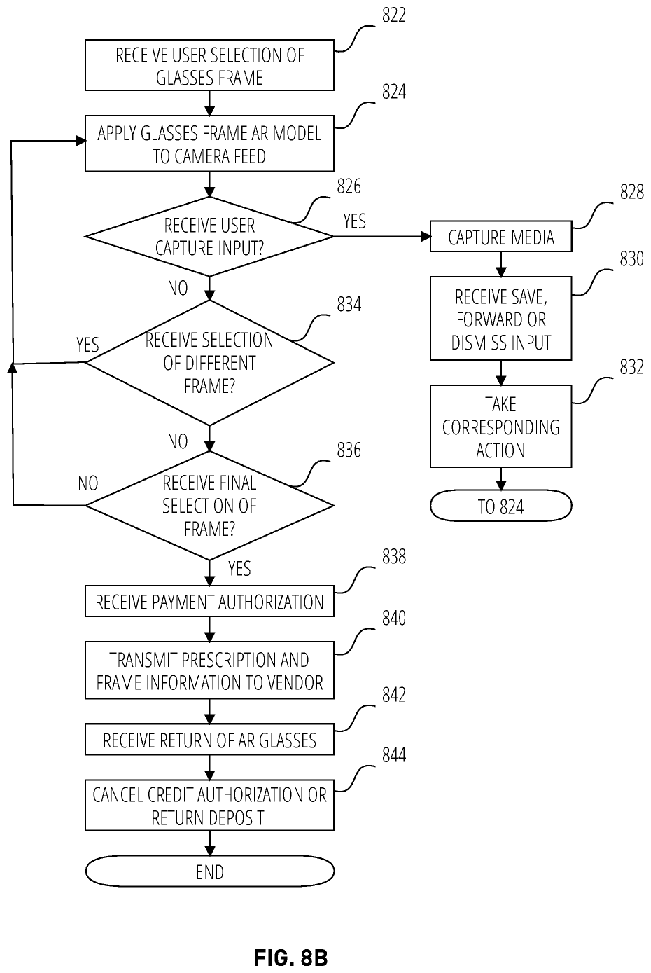

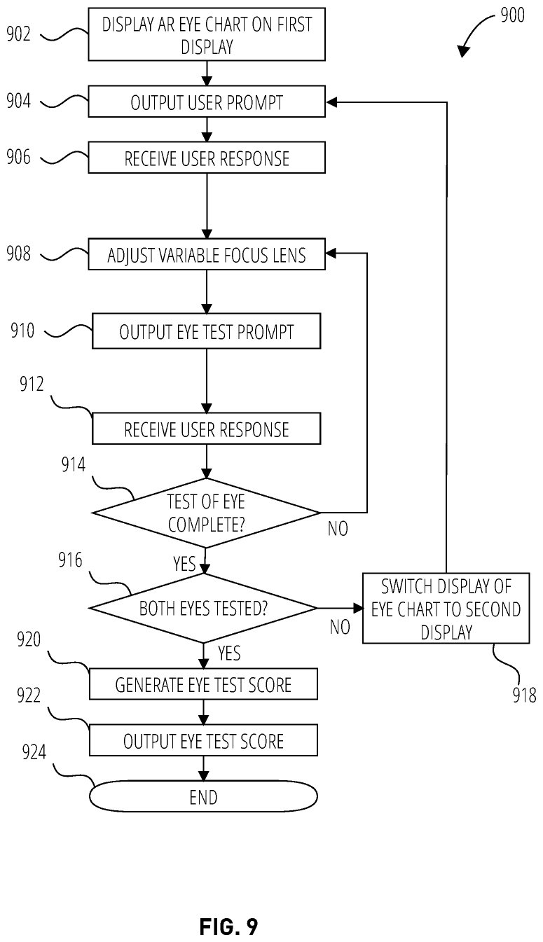

At operation 808, an eye test is performed by the messaging application 1246 in conjunction with the AR glasses 600 to determine the user's glasses prescription. In one example, the eye test is performed as described below with reference to FIG. 9. In another example, additional eye tests and assessments and associated information may be offered for user selection as described below with reference to FIG. 10.

Upon completion of the eye test in operation 818, the results (comprising for example a “sphere” lens power parameter for nearsighted or farsighted vision and “cylinder” and “axis” parameters to correct astigmatism) are stored by the messaging system 100. The results may also be displayed to the user for information, saving or forwarding. Additional options (such as lens color/tint, bifocal/trifocal/progressive lenses etc.) may be provided for user selection at some point throughout the process, for example upon completion of the eye test in operation 818 or upon final frame selection in operation 836.

In operation 820, the messaging application 1246 displays a prescription glasses frame user interface or an e-store storefront, allowing the user to browse and select a glasses frame. The storefront may be associated with a particular brand or may provide viewing and selection of a number of different brands. The storefront provides a selection of frames from which the user can choose, filterable and sortable by any relevant parameter such as gender identity, brand, price, color, size, shape, frame material, and so forth.

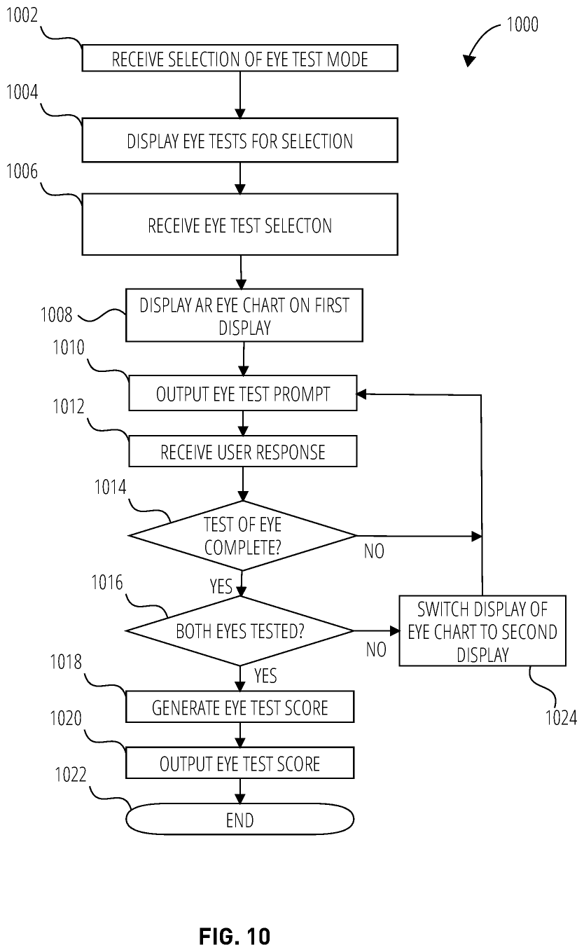

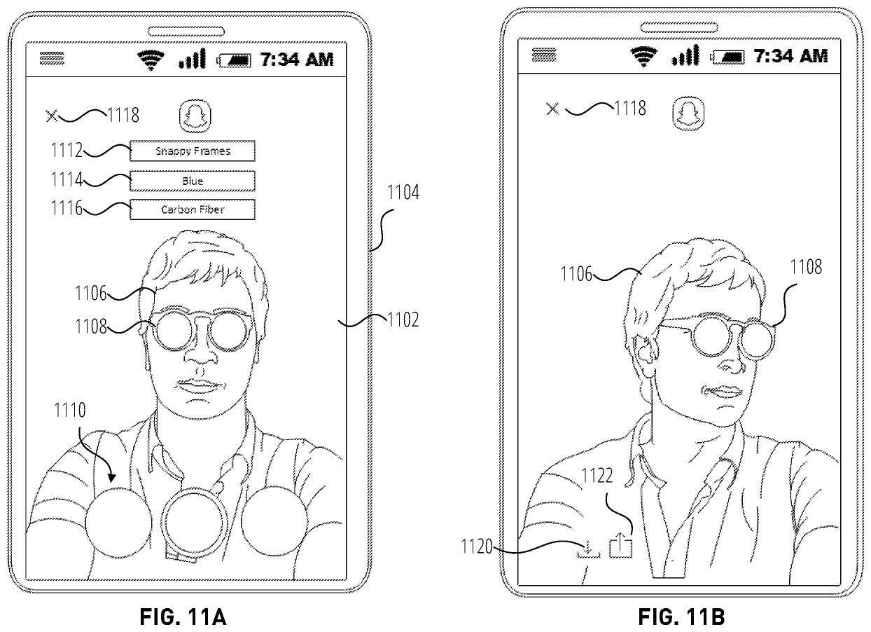

Upon receipt of user selection of a particular frame in operation, the messaging application 1246 retrieves or loads, and applies an AR model of the selected frame to the image of the user in the video feed from the front-facing camera, and displays it on the display of the user device 102, as shown in more detail in FIG. 11A and FIG. 11B. At this point, the user can provide image or video capture input at operation 826, for example by tapping or tapping and holding an image capture icon for still image or video capture respectively. In response, the messaging application 1246 captures the modified video feed with the AR model of the currently-selected frame shown on the face of the user in operation 828.

The user is then given the option to save, forward or dismiss the captured media in operation 830, and the corresponding action is taken by the messaging application 1246 in operation 832. Forwarding of the media may include posting the captured media to a social networking service, sending it in a chat session, and so forth. After the corresponding action in operation 832 has been completed, the method returns to operation 824, in which the selected frames are shown on the user's face.

The user messaging application 1246 may again receive user capture input at operation 826, or alternatively the messaging application may receive selection of a different frame at operation 834, if so, the method returns to operation 824, in which the AR model of the newly-selected frame is applied to the image of the user in the video feed from the front-facing camera, and displays it on the display of the user device 102 as before.

In operation 836, the messaging application 1246 receives final user selection of a frame. At this time, payment authorization (and payment information if not already received) is obtained at operation 838. The prescription and frame information is then transmitted to one or more vendors in operation 840. For example, the frame may be ordered and shipped from a frame vendor to a lens grinding vendor, who then grinds and fits the lenses to the frame before shipping them to the user. In one example, the provider of the messaging system 100 may receive a commission or referral or service fee for providing the services described herein.

If AR glasses 600 have been shipped to the user for temporary use, upon receipt of their return at operation 842, the corresponding credit authorization is cancelled or the deposit returned/refunded at operation 844, minus any rental or other charges, at which point the method ends.

A number of variations of the flowchart 800 are possible. In one example, the user may already be in possession of their prescription information, for example provided by an optometrist who has already performed a traditional eye test. In such case, the user will input the prescription information into the messaging application 1246 instead of the test being performed using the AR glasses 600. In such a case, the flowchart 800 will be substantially as shown in FIG. 8B. Similarly, the user may also browse and save or “favorite” a frame before obtaining prescription information with which to place an order, for example from an optometrist performing a traditional eye test.

In another example, possibly depending on the legal requirements in the country in question, an optometrist may need to be involved in the eye test. In such a case, the user may be presented with a list of participating optometrists from which to select a supervising optometrist. Upon receipt of selection of an optometrist, the selected optometrist's availability may be displayed and scheduling options presented to the user. Selected scheduling options may then be transmitted to the selected optometrist and calendared by the messaging application 1246.

When the appointed time arrives, the user will don the AR glasses 600, and commence a joint communication session with the optometrist on the optometrist's messaging application 1246 running on a user device 102. In one example, the user and the optometrist may be joined in a video chat session on the client device, and the optometrist can observe the performance of the eye test(s) via a video feed provided from a front-facing camera of the user's user device 102, suitably held or positioned to show the user's face while the eye test is performed. In another example, a shared session is created in which a view of the AR eye chart 704, the audio provided to the user (test prompts) and captured from user (test responses) by the AR glasses 300 or the user device 102, are transmitted to and rendered on the optometrist's user device 102. This may be in addition to or instead of a video feed from the front facing camera on the user's client device 1104.

In yet another example, summary information gathered during the eye test may be transmitted to a selected optometrist's user device 102, for the optometrist to view and approve after the eye test has been completed using the AR glasses 600 as described above. The summary information may for example be the eye charts presented to the user, a text version of the prompts and responses, and the prescription information derived therefrom.

Upon completion of the test and review by the optometrist, an electronic signature, certificate or other authorization may be provided by the optometrist from the optometrist's messaging application 1246 and user device 102 to the user's client device, permitting placement of the order by the user from the user's user device 102 as described above. Payment to the optometrist for the services rendered may be included as part of operation 838.

FIG. 9 is a flowchart 900 illustrating a process for determining a user's eyeglasses prescription, in accordance with one example. For explanatory purposes, the operations of the flowchart 900 are described herein as occurring in serial, or linearly. However, multiple operations of the flowchart 900 may occur in parallel. In addition, the operations of the flowchart 900 need not be performed in the order shown and/or one or more blocks of the flowchart 900 need not be performed and/or can be replaced by other operations.

The operations illustrated in FIG. 9 will typically execute on client device 428 in an application such as messaging application 1246 or a dedicated eye test application. For the purposes of clarity, flowchart 900 is discussed herein with reference to such an example. Various implementations are of course possible, with some of the operations taking place in server system 432, or with one application calling another application or SDK for required functionality. In one example, the operations are performed jointly between messaging application 1246 running on the client device 428 and the data processor 402 and associated hardware in or associated with the AR glasses 600.

The method starts at operation 902, which will commence after receipt of selection of a menu item in operation 1006 in FIG. 10 corresponding to determining a user's eyeglasses prescription. In some cases, the method of FIG. 10 will be performed initially to establish a baseline visual acuity score for the user, if not done already, before commencing with the method of FIG. 9. At operation 902, prescription determination then commences at operation 902 with the messaging application 1246 displaying the AR eye chart 704 on a first of the displays 410 corresponding to a first of the user's eyes at operation 902. The AR eye chart 704 is displayed as discussed above. At this time, the second of the displays will likely display nothing (for example provide an unobstructed view) or may display a solid color (such as black) or other effect to obscure the other eye. In the case of a VR headset, the other display may display the virtual reality environment in which the user finds themselves or may display a solid color (such as black) or other effect to obscure the eye.

An initial user prompt may be provided at this time at operation 904, to direct a person to one or more features of the chart. For example, “Look at the smallest line in the chart that you can read.” The user prompt may include an instruction such as “Say ‘yes’ when done or ‘Exit’ to exit.” The user prompt may be an audible prompt provided via a speaker in the client device 428, the AR glasses 600, or a connected loudspeaker such as wired or wireless earbuds or a headset.

If an affirmative user response has been required in operation 904, the messaging application 1246 will wait to receive it in operation 906 before proceeding with the method at operation 908. If a different response is received (such as “exit”) then the messaging application 1246 takes the appropriate action, such as to return to the previous or an earlier menu or interface.

At operation 908, the variable focus lens 602 corresponding to the eye under test is then adjusted by the messaging application 1246 to provide a change in optical power as is known in tests for determining eyeglasses prescriptions. At operation 910, the messaging application 1246 provides a relevant user prompt, for example whether the change in optical power performed in operation 908 makes the user's vision better or worse or whether there is no change.

At operation 912, the user's response is received by the messaging application 1246. The user's response may for example be an audible response received by a microphone in the client device 428, the AR glasses 600, or a connected microphone for example such as in wired or wireless earbuds or a headset. Voice recognition is performed on the user's responses and the responses (for example “Better,” “Worse” or “The same”) are provided as input to the messaging application 1246, which then determines the next adjustment of the variable focus lens 602 for the eyeglasses prescription test as is known in the art.

In another example, operation 908 and operation 910 may be performed in a loop while waiting for a user response, such that the variable focus lens 602 toggles (at comfortable intervals) between the previous optical power and the new optical power. In such a case, user prompts are synchronized with the change between the two optical powers, requesting that the user indicate a preference between the two optical powers as they are generated, for example: “Is this one better? Or is this one better? Or are they the same?”

At operation 914, after receiving a valid user response, the messaging application 1246 determines whether or not the test of the eye under consideration is complete. The test may for example be complete if further adjustments to the particular variable focus lens 602 does not result in any further improvement in the user's vision for the eye under test. If the test is not complete, the method returns to operation 910 where the next prompt is provided by the messaging application 1246.

The completeness of the test may occur when a single test is complete, or, in the case of multiple tests, completion of the first test may result in the commencement of a second or further test on the same eye. For example, completion of the display of a visual acuity chart, user prompts and variable focus lens 602 adjustments to determine a “sphere” lens power parameter for nearsighted or farsighted vision may result in the commencement of the display of charts, user prompts and variable focus lens 602 adjustments to determine “cylinder” and “axis” parameters to correct astigmatism. In such a case, the method returns to operation 908 for the second or further tests to be performed, with appropriate modifications in the charts displayed, the prompts provided and the user responses that are expected.

If the messaging application 1246 determines that the test(s) of the eye under consideration is/are complete, the method proceeds to operation 916 where the messaging application 1246 checks whether only one eye has been tested or whether both eyes have been tested. If only one eye has been tested, the method proceeds at operation 918 where the contents of the displays are switched such that the AR eye chart 704 is switched to the other display. The letters used in the eye chart are preferably also changed at this time to prevent the user from relying on their memory of the original AR eye chart 704. Any obscuring or other display content or color shown to the eye that was previously not under test is switched to the eye for which the test has just been completed.

The flowchart 900 then continues as before through operation 910 to operation 914 to test the other eye. After both eyes have been tested, as verified at operation 916, the messaging application 1246 generates an eye test score at operation 920 using known methods, based on the adjustments to the variable focus lenses 602 and the user's responses thereto. The eye test score may be an eyeglasses prescription including sphere, cylinder and axis parameters for both left and right eyes.

In one example, the cameras 512 also capture one or more images of the user's eyes during or after performance of the eye tests discussed above. Since the positions and characteristics of the cameras 512 are known, after the user's pupils have been detected in the image(s) using object recognition image processing techniques, the user's interpupillary distance can be determined.

The eye test score is then output at operation 922. This may be done by displaying the results of the eye test on the AR glasses 600, by displaying the results on a display of the client device 428, audibly, or by any other means. The results may also be compared against previous test results to illustrate any degradation. The results and the underlying, intermediate or associated data (such as the contents of the AR eye charts 704, user responses and a comparison of same, any determined interpupillary distance) may be transmitted to an eye specialist for review or record-keeping. The user may also be given options to save the test results, post them to a social networking site, or transmit them in a chat session.

The flowchart 900 then ends at operation 924. The messaging application 1246 may then for example revert to a default user interface for the messaging applications 1246.

FIG. 10 is a flowchart 1000 illustrating a process for conducting an AR eye test, in accordance with one example. For explanatory purposes, the operations of the flowchart 1000 are described herein as occurring in serial, or linearly. However, multiple operations of the flowchart 1000 may occur in parallel. In addition, the operations of the flowchart 1000 need not be performed in the order shown and/or one or more blocks of the flowchart 1000 need not be performed and/or can be replaced by other operations.

The operations illustrated in FIG. 10 will typically execute on client device 428 in an application such as messaging application 1246 or a dedicated eye test application. For the purposes of clarity, flowchart 1000 is discussed herein with reference to such an example. Various implementations are of course possible, with some of the operations taking place in server system 432, or with one application calling another application or SDK for required functionality. In one example, the operations are performed jointly between messaging application 1246 running on the client device 428 and the data processor 402 and associated hardware in or associated with the AR glasses 600, or AR glasses 300 or AR glasses 500 as appropriate.

The method starts at operation 1002 with the messaging application 1246 receiving user input corresponding to selection of an eye test mode. In response, the messaging application 1246 displays an eye test user interface on one or both of the displays 410 or on the display of the client device 428 at operation 1004. The user interface provides one or more options relating to eye tests, for example to view previous test results, options for selecting different eye tests to be performed using the AR glasses 600, and so forth.

In one example, the user interface may provide a menu or other user interface display of visual or ophthalmic tests from which a user may choose, including for example the ophthalmic imaging tests described in US Provisional Patent Application entitled “Ophthalmic Imaging Using a Head-Worn Device” filed concurrently herewith, Attorney Docket No. 4218.C76PRV, the contents of which are incorporated herein by reference as if explicitly set forth, as well as the determination of a user's eyeglasses prescription as described below with reference to FIG. 9.

Selection of a visual acuity test is then received by the messaging application 1246 at operation 1006. This can be done using any selection technique, including by receiving a touch input on client device 428, voice input, gesture recognition, or the like.

The visual acuity test then commences at operation 1008 with the messaging application 1246 displaying the AR eye chart 704 on a first of the displays 410 corresponding to a first of the user's eyes at operation 1008. The AR eye chart 704 is displayed as discussed above. At this time, the second of the displays may display nothing (for example provide an unobstructed view) or may display a solid color (such as black) or other effect to obscure the other eye. In the case of a VR headset, the other display may display the virtual reality environment in which the user finds themselves or may display a solid color (such as black) or other effect to obscure the eye.

At operation 1010, the messaging application 1246 provides a relevant user prompt. The user prompt may be an audible prompt provided via a speaker in the client device 428, the AR glasses 600, or a connected loudspeaker such as wired or wireless earbuds or a headset. The user prompt may for example instruct the user to “Read the Xth line of the display,” where X is the line of the AR chart under consideration. The eye test prompt may also vary depending on the user's interactions. For example, if a user has made mistakes in one line, the prompt to read that line may be repeated.

At operation 1012, the user's response is received by the messaging application 1246. The user's response may for example be an audible response received by a microphone in the client device 428, the AR glasses 600, or a connected microphone for example such as in wired or wireless earbuds or a headset. Voice recognition is performed on the user's responses and the responses are correlated with the corresponding letter in the AR eye chart 704. For example, the first response will be associated with the first letter in the row under consideration in AR eye chart 704, the second response will be associated with the second letter in the row under consideration, and so forth, to permit scoring of the user's responses by comparing the responses to the letters.

At operation 1014, the messaging application 1246 determines whether or not the test of the eye under consideration is complete. The test may for example be complete if all rows of the AR eye chart 704 have been considered or if a threshold number of errors in one row has been exceeded. If the test is not complete, the method returns to operation 1010 where the next prompt is provided by the messaging application 1246.

If the messaging application 1246 determines that the test of the eye under consideration is complete, the method proceeds to operation 1016 where the messaging application 1246 checks whether only one eye has been tested or whether both eyes have been tested. If only one eye has been tested, the method proceeds at operation 1024 where the content of the displays are switched such that the AR eye chart 704 is switched to the other display. The letters used in the eye chart are preferably also changed at this time to prevent the user from relying on their memory of the original AR eye chart 704. Any obscuring or other display content or color shown to the eye that was previously not under test is switched to the eye for which the test has just been completed.

The flowchart 1000 then continues as before through operation 1010 to operation 1014 to test the other eye. After both eyes have been tested, as verified at operation 1016, the messaging application 1246 generates an eye test score at operation 1018 using known methods, by comparing the contents of the AR eye chart 704 with the user's responses.

The eye test score is then output at operation 1020. This may be done by displaying the results of the eye test on the AR glasses 600, by displaying the results on a display of the client device 428, audibly, or by any other means. The results may also be compared against previous test results to illustrate any degradation. The results and the underlying or intermediate data (e.g. the contents of the AR eye charts 704, user responses and a comparison of same) may be transmitted to an eye specialist for review or record-keeping. The user may also be given options to save the test results, post them to a social networking site, or transmit them in a chat session.

The flowchart 1000 then ends at operation 1022. The messaging application 1246 may then for example revert to a default user interface for the messaging applications 1246.

FIG. 11A is an example of a user interface that is displayed on a user device 102 in response to receiving user selection of a glasses frame in operation 822 in flowchart 800. As can be seen, an image of a user 1106 is displayed on the display 1102 of a client device 1104, with an AR glasses frame 1108 corresponding to the selected frame displayed on the face of the user 1106. The illustrated image is part of a live feed captured by a front-facing camera of the client device 1104, and the AR glasses frame 1108 are fixed relative to the user's face, so that the user can change their position to view or capture an image or video of them wearing the glasses from different angles, as shown in FIG. 11B.

In one example, a horizontally-scrollable carousel 1110 is provided, in which different frames are presented in thumbnail version (not illustrated) in each of the circular carousel elements. The currently-selected glasses frame is located in the central carousel element, and another frame can be selected by scrolling the carousel left or right so that its thumbnail occupies the central position, at which point its AR glasses frame 1108 will be displayed on the user's face.

Dropdown menu items may also be provided to allow the selection of available glasses to be filtered by one or more criteria. For example, user interface elements corresponding to a brand selection menu 1112, a color selection menu 1114 and a frame material selection menu 1116 may be provided. Touching the relevant user interface item will allow the user to select between “all” or specific brands, colors or materials. Additional options may also be provided.

A dismiss icon 1118 may be provided to return the user to the storefront for the glasses frames.

FIG. 11B is an example of a user interface that may be displayed in response to capture input, for example a tap or a long press on the central carousel element. The captured media is previewed, and can be dismissed by receipt of a tap on dismiss icon 1118 (to return to the user interface shown in FIG. 11A), or saved by receipt of a tap on save icon 1120, or forwarded based on receipt of a tap on forward icon 1122. Selection of forward icon 1122 provides further options for sharing the captured media in a chat session, on a social network activity feed, and so forth.

FIG. 12 is a block diagram 1200 illustrating a software architecture 1204, which can be installed on any one or more of the devices described herein. The software architecture 1204 is supported by hardware such as a machine 1202 that includes processors 1220, memory 1226, and I/O components 1238. In this example, the software architecture 1204 can be conceptualized as a stack of layers, where each layer provides a particular functionality. The software architecture 1204 includes layers such as an operating system 1212, libraries 1208, frameworks 1210, and applications 1206. Operationally, the applications 1206 invoke API calls 1250 through the software stack and receive messages 1252 in response to the API calls 1250.

The operating system 1212 manages hardware resources and provides common services. The operating system 1212 includes, for example, a kernel 1214, services 1216, and drivers 1222. The kernel 1214 acts as an abstraction layer between the hardware and the other software layers. For example, the kernel 1214 provides memory management, processor management (e.g., scheduling), component management, networking, and security settings, among other functionality. The services 1216 can provide other common services for the other software layers. The drivers 1222 are responsible for controlling or interfacing with the underlying hardware. For instance, the drivers 1222 can include display drivers, camera drivers, BLUETOOTH® or BLUETOOTH® Low Energy drivers, flash memory drivers, serial communication drivers (e.g., Universal Serial Bus (USB) drivers), WI-FI® drivers, audio drivers, power management drivers, and so forth.

The libraries 1208 provide a low-level common infrastructure used by the applications 1206. The libraries 1208 can include system libraries 1218 (e.g., C standard library) that provide functions such as memory allocation functions, string manipulation functions, mathematic functions, and the like. In addition, the libraries 1208 can include API libraries 1224 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 1208 can also include a wide variety of other libraries 1228 to provide many other APIs to the applications 1206.

The frameworks 1210 provide a high-level common infrastructure that is used by the applications 1206. For example, the frameworks 1210 provide various graphical user interface (GUI) functions, high-level resource management, and high-level location services. The frameworks 1210 can provide a broad spectrum of other APIs that can be used by the applications 1206, some of which may be specific to a particular operating system or platform.

In an example, the applications 1206 may include a home application 1236, a contacts application 1230, a browser application 1232, a book reader application 1234, a location application 1242, a media application 1244, a messaging application 1246, a game application 1248, and a broad assortment of other applications such as third-party applications 1240. The applications 1206 are programs that execute functions defined in the programs. Various programming languages can be employed to create one or more of the applications 1206, structured in a variety of manners, such as object-oriented programming languages (e.g., Objective-C, Java, or C++) or procedural programming languages (e.g., C or assembly language). In a specific example, the third-party applications 1240 (e.g., applications developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party applications 1240 can invoke the API calls 1250 provided by the operating system 1212 to facilitate functionality described herein.

FIG. 13 is a diagrammatic representation of a machine 1300 within which instructions 1310 (e.g., software, a program, an application, an applet, an app, or other executable code) for causing the machine 1300 to perform any one or more of the methodologies discussed herein may be executed. For example, the instructions 1310 may cause the machine 1300 to execute any one or more of the methods described herein. The instructions 1310 transform the general, non-programmed machine 1300 into a particular machine 1300 programmed to carry out the described and illustrated functions in the manner described. The machine 1300 may operate as a standalone device or may be coupled (e.g., networked) to other machines. In a networked deployment, the machine 1300 may operate in the capacity of a server machine or a client machine in a server-client network environment, or as a peer machine in a peer-to-peer (or distributed) network environment. The machine 1300 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1310, sequentially or otherwise, that specify actions to be taken by the machine 1300. Further, while only a single machine 1300 is illustrated, the term “machine” shall also be taken to include a collection of machines that individually or jointly execute the instructions 1310 to perform any one or more of the methodologies discussed herein.

The machine 1300 may include processors 1302, memory 1304, and I/O components 1306, which may be configured to communicate with each other via a bus 1344. In an example, the processors 1302 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1308 and a processor 1312 that execute the instructions 1310. The term “processor” is intended to include multi-core processors that may comprise two or more independent processors (sometimes referred to as “cores”) that may execute instructions contemporaneously. Although FIG. 13 shows multiple processors 1302, the machine 1300 may include a single processor with a single core, a single processor with multiple cores (e.g., a multi-core processor), multiple processors with a single core, multiple processors with multiples cores, or any combination thereof.

The memory 1304 includes a main memory 1314, a static memory 1316, and a storage unit 1318, both accessible to the processors 1302 via the bus 1344. The main memory 1304, the static memory 1316, and storage unit 1318 store the instructions 1310 embodying any one or more of the methodologies or functions described herein. The instructions 1310 may also reside, completely or partially, within the main memory 1314, within the static memory 1316, within machine-readable medium 1320 within the storage unit 1318, within at least one of the processors 1302 (e.g., within the processor's cache memory), or any suitable combination thereof, during execution thereof by the networked system 400.

The I/O components 1306 may include a wide variety of components to receive input, provide output, produce output, transmit information, exchange information, capture measurements, and so on. The specific I/O components 1306 that are included in a particular machine will depend on the type of machine. For example, portable machines such as mobile phones may include a touch input device or other such input mechanisms, while a headless server machine will likely not include such a touch input device. It will be appreciated that the I/O components 1306 may include many other components that are not shown in FIG. 13. In various examples, the I/O components 1306 may include output components 1328 and input components 1332. The output components 1328 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1332 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further examples, the I/O components 1306 may include biometric components 1334, motion components 1336, environmental components 1338, or position components 1340, among a wide array of other components. For example, the biometric components 1334 include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1336 include acceleration sensor components (e.g., accelerometer), gravitation sensor components, rotation sensor components (e.g., gyroscope), and so forth. The environmental components 1338 include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1340 include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1306 further include communication components 1342 operable to couple the networked system 400 to a network 1322 or devices 1324 via a coupling 1330 and a coupling 1326, respectively. For example, the communication components 1342 may include a network interface component or another suitable device to interface with the network 1322. In further examples, the communication components 1342 may include wired communication components, wireless communication components, cellular communication components, Near Field Communication (NFC) components, Bluetooth® components (e.g., Bluetooth® Low Energy), WiFi® components, and other communication components to provide communication via other modalities. The devices 1324 may be another machine or any of a wide variety of peripheral devices (e.g., a peripheral device coupled via a USB).

Moreover, the communication components 1342 may detect identifiers or include components operable to detect identifiers. For example, the communication components 1342 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1342, such as location via Internet Protocol (IP) geolocation, location via Wi-Fi® signal triangulation, location via detecting an NFC beacon signal that may indicate a particular location, and so forth.

The various memories (e.g., memory 1304, main memory 1314, static memory 1316, and/or memory of the processors 1302) and/or storage unit 1318 may store one or more sets of instructions and data structures (e.g., software) embodying or used by any one or more of the methodologies or functions described herein. These instructions (e.g., the instructions 1310), when executed by processors 1302, cause various operations to implement the disclosed examples.

The instructions 1310 may be transmitted or received over the network 1322, using a transmission medium, via a network interface device (e.g., a network interface component included in the communication components 1342) and using any one of a number of well-known transfer protocols (e.g., hypertext transfer protocol (HTTP)). Similarly, the instructions 1310 may be transmitted or received using a transmission medium via the coupling 1326 (e.g., a peer-to-peer coupling) to the devices 1324.

A “carrier signal” refers to any intangible medium that is capable of storing, encoding, or carrying instructions for execution by the machine, and includes digital or analog communications signals or other intangible media to facilitate communication of such instructions. Instructions may be transmitted or received over a network using a transmission medium via a network interface device.

A “client device” refers to any machine that interfaces to a communications network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network.

A “communication network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long-range protocols, or other data transfer technology.

A “component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various examples, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application specific integrated circuit (ASIC). A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering examples in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In examples in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some examples, the processors or processor-implemented components may be located in a single geographic location (e.g., within a home environment, an office environment, or a server farm). In other examples, the processors or processor-implemented components may be distributed across a number of geographic locations.

A “computer-readable medium” refers to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

An “ephemeral message” refers to a message that is accessible for a time-limited duration. An ephemeral message may be a text, an image, a video and the like. The access time for the ephemeral message may be set by the message sender. Alternatively, the access time may be a default setting or a setting specified by the recipient. Regardless of the setting technique, the message is transitory.

A “machine-storage medium” refers to a single or multiple storage devices and/or media (e.g., a centralized or distributed database, and/or associated caches and servers) that store executable instructions, routines and/or data. The term shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

A “processor” refers to any circuit or virtual circuit (a physical circuit emulated by logic executing on an actual processor) that manipulates data values according to control signals (e.g., “commands”, “op codes”, “machine code”, and so forth) and which produces corresponding output signals that are applied to operate a machine. A processor may, for example, be a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC) or any combination thereof. A processor may further be a multi-core processor having two or more independent processors (sometimes referred to as “cores”) that may execute instructions contemporaneously.

A “signal medium” refers to any intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

Changes and modifications may be made to the disclosed examples without departing from the scope of the present disclosure. These and other changes or modifications are intended to be included within the scope of the present disclosure, as expressed in the following claims.

### Glossary

“Carrier signal” refers to any intangible medium that is capable of storing, encoding, or carrying instructions for execution by the machine, and includes digital or analog communications signals or other intangible media to facilitate communication of such instructions. Instructions may be transmitted or received over a network using a transmission medium via a network interface device.

“Client device” refers to any machine that interfaces to a communications network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network.

“Communication network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long-range protocols, or other data transfer technology.

“Component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various examples, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application specific integrated circuit (ASIC). A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering examples in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In examples in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some examples, the processors or processor-implemented components may be located in a single geographic location (e.g., within a home environment, an office environment, or a server farm). In other examples, the processors or processor-implemented components may be distributed across a number of geographic locations.

“Computer-readable storage medium” refers to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

“Ephemeral message” refers to a message that is accessible for a time-limited duration. An ephemeral message may be a text, an image, a video and the like. The access time for the ephemeral message may be set by the message sender. Alternatively, the access time may be a default setting or a setting specified by the recipient. Regardless of the setting technique, the message is transitory.

“Machine storage medium” refers to a single or multiple storage devices and media (e.g., a centralized or distributed database, and associated caches and servers) that store executable instructions, routines and data. The term shall accordingly be taken to include, but not be limited to, solid-state memories, and optical and magnetic media, including memory internal or external to processors. Specific examples of machine-storage media, computer-storage media and device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

“Non-transitory computer-readable storage medium” refers to a tangible medium that is capable of storing, encoding, or carrying the instructions for execution by a machine.

“Signal medium” refers to any intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

What is claimed is:

1. A method, executed by one or more processors, of providing prescription glasses to a user according to their vision prescription, the method comprising:
performing an eye test using a display device of a head-worn device to determine the user's vision prescription;
displaying a selection of glasses frames on a portable device;
receiving user selection of a glasses frame using the portable device;
displaying an AR model of the selected glasses frame on an image of the user captured by a camera of the portable device;
receiving final selection of a glasses frame; and
transmitting the user's vision prescription and an identifier of the finally selected glasses frame to at least one vendor of optical prescription services.

2. The method of claim 1, further comprising:
providing a prompt via the portable device relating to whether or not the user has access to a head-worn device; and
in response to receiving user input indicating that the user does not have access to a head-worn device, transmitting instructions to ship a head-worn device to the user.

3. The method of claim 2, further comprising:
obtaining a payment authorization prior to transmitting the instructions to ship a head-worn device to the user.

4. The method of claim 3, further comprising:
upon receiving notification of return of the head-worn device from the user, canceling or refunding the payment authorization.

5. The method of claim 1, further comprising:
receiving a capture-media input while the AR model is being displayed on an image of the user;
capturing media corresponding to the display of the AR model on the image of the user;
receiving a forward-media input; and
transmitting the captured media in a message or chat session.

6. The method of claim 1, further comprising:
creating a joint communication session with a remote device associated with an eye-care practitioner;
transmitting data relating to the eye test being performed using the display device of a head-worn device to the remote device; and
receiving approval data for the user's vision prescription from the remote device.

7. The method of claim 1, further comprising:
transmitting summary data, generated during the performance of the eye test using the display device of a head-worn device eye test, to a remote device associated with an eye-care practitioner; and
receiving approval data for the user's vision prescription from the remote device.

8. The method of claim 1, further comprising:
displaying a user interface element listing characteristics of prescription glasses frames;
receiving user selection of one or more specific characteristics; and
limiting the selection of glasses frames on the portable device by the specific characteristics.

9. The method of claim 8, further comprising:
displaying identifiers of the limited selection of glasses frames in a carousel user interface;
receiving touch input to place a particular glasses frame identifier in a central position in the carousel user interface; and
displaying an AR model of the particular glasses frame on an image of the user captured by a camera of the portable device.

10. A computer system comprising:
one or more processors; and
a memory storing instructions that, when executed by the one or more processors, configure the computer system to provide prescription glasses to a user according to their vision prescription by performing operations comprising:
performing an eye test using a display device of a head-worn device to determine the user's vision prescription;
displaying a selection of glasses frames on a portable device;
receiving user selection of a glasses frame using the portable device;
displaying an AR model of the selected glasses frame on an image of the user captured by a camera of the portable device;
receiving final selection of a glasses frame; and
transmitting the user's vision prescription and an identifier of the finally selected glasses frame to at least one vendor of optical prescription services.

11. The computer system of claim 10, wherein the operations further comprise:
receiving a capture-media input while the AR model is being displayed on an image of the user;
capturing media corresponding to the display of the AR model on the image of the user;
receiving a forward-media input; and
transmitting the captured media in a message or chat session.

12. The computer system of claim 10, wherein the operations further comprise:
creating a joint communication session with a remote device associated with an eye-care practitioner;
transmitting data relating to the eye test being performed using the display device of a head-worn device to the remote device; and
receiving approval data for the user's vision prescription from the remote device.

13. The computer system of claim 10, wherein the operations further comprise:
transmitting summary data, generated during the performance of the eye test using the display device of a head-worn device eye test, to a remote device associated with an eye-care practitioner; and
receiving approval data for the user's vision prescription from the remote device.

14. The computer system of claim 10, wherein the operations further comprise:
displaying identifiers of a limited selection of glasses frames in a carousel user interface;
receiving touch input to place a particular glasses frame identifier in a central position in the carousel user interface; and
displaying an AR model of the particular glasses frame on an image of the user captured by a camera of the portable device.

15. A non-transitory computer-readable storage medium, the computer-readable storage medium including instructions that when executed by a computer system, cause the computer system to provide prescription glasses to a user according to their vision prescription by performing operations comprising:
performing an eye test using a display device of a head-worn device to determine the user's vision prescription;
displaying a selection of glasses frames on a portable device;
receiving user selection of a glasses frame using the portable device;
displaying an AR model of the selected glasses frame on an image of the user captured by a camera of the portable device;
receiving final selection of a glasses frame; and
transmitting the user's vision prescription and an identifier of the finally selected glasses frame to at least one vendor of optical prescription services.

16. The non-transitory computer-readable storage medium of claim 15, wherein the operations further comprise:
receiving a capture-media input while the AR model is being displayed on an image of the user;
capturing media corresponding to the display of the AR model on the image of the user;
receiving a forward-media input; and
transmitting the captured media in a message or chat session.

17. The non-transitory computer-readable storage medium of claim 15, wherein the operations further comprise:
creating a joint communication session with a remote device associated with an eye-care practitioner;
transmitting data relating to the eye test being performed using the display device of a head-worn device to the remote device; and
receiving approval data for the user's vision prescription from the remote device.

18. The non-transitory computer-readable storage medium of claim 15, wherein the operations further comprise:
transmitting summary data, generated during the performance of the eye test using the display device of a head-worn device eye test, to a remote device associated with an eye-care practitioner; and
receiving approval data for the user's vision prescription from the remote device.

19. The non-transitory computer-readable storage medium of claim 15, wherein the operations further comprise:
displaying identifiers of a limited selection of glasses frames in a carousel user interface;
receiving touch input to place a particular glasses frame identifier in a central position in the carousel user interface; and
displaying an AR model of the particular glasses frame on an image of the user captured by a camera of the portable device.

20. The non-transitory computer-readable storage medium of claim 15, wherein the operations further comprise:
providing a prompt via the portable device relating to whether or not the user has access to a head-worn device; and
in response to receiving user input indicating that the user does not have access to a head-worn device, transmitting instructions to ship a head-worn device to the user.
